(19) United States
(12) Patent Application Publication
(10) Pub. No.: US 2025/0073898 A1
(43) Pub. Date: Mar. 6, 2025

(54) INTERVENTIONAL ROBOTIC SYSTEM WITH CURVE TRACKING

(71) Applicant: Magnisity Ltd., Hod-HaSharon (IL)

(72) Inventors: Benjamin GREENBURG, Hod-HaSharon (IL); Ron BARAK, Tel-Aviv (IL); Amit COHEN, Ramat Gan (IL); Dror GARDOSH, Rishon LeTsiyon (IL); Nadav LEHRER, Givatayim (IL)

(73) Assignee: Magnisity Ltd., Hod-HaSharon (IL)

(21) Appl. No.: 18/823,814

(22) Filed: Sep. 4, 2024

Related U.S. Application Data
(60) Provisional application No. 63/536,468, filed on Sep. 4, 2023.

(51) Int. Cl. B25J 9/16

(52) U.S. Cl. CPC B25J 9/1635; B25J 9/1692

(57) ABSTRACT

A system and method for controlling an interventional robotic system, including an elongated flexible device, including a non-deflectable section in a proximal part of the device, a deflectable section in a distal part of the device, a curve sensor, configured to sense a curve of the deflectable section, and steering wires to deflect the deflectable section, and a processing module including a curve tracking module and a robot controller, configured to calculate and perform steering actions by positioning each of the steering wires in a certain state, for bringing the device to a target pose, receive curve tracking data from the curve tracking module, and use the received curve tracking data as feedback to the robot controller, to determine a difference between a current pose of the device to the target pose of the device, and adjust the state of the steering wires to decrease the calculated difference.

### RELATED APPLICATION(S)

This application claims the benefit of priority under 35 USC § 119 (e) of U.S. Provisional Patent Application No. 63/536,468 filed on Sep. 4, 2023, the contents of which are incorporated by reference as if fully set forth herein in their entirety.

### FIELD AND BACKGROUND OF THE INVENTION

Certain Electromagnetic (EM) tracking systems use magnetometers to sense low-frequency (for example, lower than 500 Hz, or lower than 1 kHz, or lower than 10 kHz, or lower than 100 kHz) EM fields for position and orientation tracking. One application of such tracking systems is for EM shape sensing of a medical device, such as a fully shape tracked endoscope.

There are some robotic systems for endoscopic procedures, that use optical shape sensing. Optical shape sensing enables calculation of an estimated shape of a device. However, the optical sensor by itself cannot provide a localization of the device, it only measures the shape of the device rather than its position and orientation in space (relative to some fixed coordinate system). Certain solutions exist to attempt and recover a position and orientation of the shape tracked device by using shape integration methods relative to certain anchors at known positions and orientation, however these methods are prone to large position and/or orientation error and poor SNR (Signal-Noise-Ratio) at the device's tip, where accuracy is most needed.

For example, US patent application No. 2018/078318 of Intuitive Surgical Operations, Inc., discloses a method performed by a computing system, that comprises receiving shape information for an elongate flexible portion of a medical instrument. The medical instrument includes a reference portion movably coupled to a fixture having a known pose in a surgical reference frame. The fixture includes a constraint structure having a known constraint structure location in the surgical reference frame. The elongated flexible portion is coupled to the reference portion and is sized to pass through the constraint structure. The method further includes receiving reference portion position information in the surgical reference frame; determining an estimated constraint structure location in the surgical reference frame from the reference portion position information and the shape information; determining a correction factor by comparing the estimated constraint structure location to the known constraint structure location; and modifying the shape information based upon the correction factor.

In U.S. Pat. No. 11,712,309, an EM curve sensor is described which consists of a sensor-array made of multiple discrete digital 3D magnetometers assembled on a Flexible Printed Circuit (FPC). The sensor-array may be embedded in an endoscope (or other tubular device) to enable EM curve tracking of that endoscope. Curve tracking provides both shape and position tracking of the device relative to a fixed coordinate system (the EM transmitter's coordinate system).

### SUMMARY OF THE INVENTION

An aspect of some embodiments of the present disclosure provides a system for controlling an interventional robotic system, including: an elongated flexible device, including: a non-deflectable section in a proximal part of the device; a deflectable section in a distal part of the device; a curve sensor, configured to sense a curve of the deflectable section; and steering wires to deflect the deflectable section; and a processing module comprising: a curve tracking module; and a robot controller, wherein the processing module is configured to: calculate and perform steering actions, according to a current calibration state, by positioning each of the steering wires in a certain state, for bringing the device to a target pose; receive curve tracking data from the curve tracking module; and use the received curve tracking data as feedback to the robot controller, to determine a difference between a current pose of the device to the target pose of the device, and adjust the state of the steering wires to decrease the calculated difference.

In some embodiments, the steering wires are calibrated to achieve a zero-tension state, before a procedure or during a procedure.

In some embodiments, the steering wires are calibrated dynamically during a procedure, to achieve and maintain a static zero-tension state.

In some embodiments, the robot controller performs a closed-loop control of the device tip deflection.

In some embodiments, the robot controller uses a predictive model of deflection, to control the device tip deflection.

In some embodiments, the deflection control is dynamically calibrated.

In some embodiments, the device includes at least two steering wires.

In some embodiments, the device includes four steering wires.

In some embodiments, the curve tracking module computes the curve by using a 6DOF electromagnetic curve sensor.

In some embodiments, the curve tracking module computes the curve by using an optical fiber sensor fixed at a distal tip of the device.

Another aspect of some embodiments of the present disclosure provides a method for controlling an interventional robotic system, including: calculating and performing by a robot controller steering actions, according to a current control calibration state, by positioning each of a plurality of steering wires in a certain state, for bringing a device to a target pose; receiving curve tracking data of the device from a curve tracking module; and using the received curve tracking data as feedback to the robot controller, to determine a difference between a current pose of the device to the target pose of the device, and adjust the state of the steering wires to decrease the calculated difference.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention.

It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of an interventional robotic system 100, according to some embodiments of the present disclosure;

FIG. 2 is a cross-sectional view 200 of device 14, according to some embodiments of the present disclosure;

FIG. 3 is a flowchart illustrating a method 300 for controlling an interventional robotic system, according to some embodiments of the present disclosure; and

FIGS. 4A and 4B are additional illustrations of steering wires 17 used to deflect a deflectable portion 14b of device 14.

### DESCRIPTION OF SPECIFIC EMBODIMENTS OF THE INVENTION

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present disclosure provides a system for controlling an interventional (e.g. endoscopic, endovascular, neurovascular, or endoluminal) robotic system, comprising: an elongated flexible device, comprising: an non-deflectable section in a proximal part of the device; a deflectable section in a distal part of the device; a curve sensor, configured to sense a curve (position and shape) of the deflectable section; and steering wires to deflect the deflectable section; and a processing module comprising: a curve tracking unit; and a robot controller, wherein the processing module is configured to: calculate and perform steering actions by positioning each of the steering wires in a certain state, for example relative to a static or dynamic zero-tension state, required to bring the device to a requested pose, according to a current calibration state; receive curve tracking data from the curve tracking module; and use the received curve tracking data as feedback to the robot controller, to determine a difference between a current pose of the device to the required pose of the device, and adjust the state of the steering wires to decrease the calculated difference.

The term “curve” throughout the present disclosure means a state that includes both a position and a shape of an elongated device.

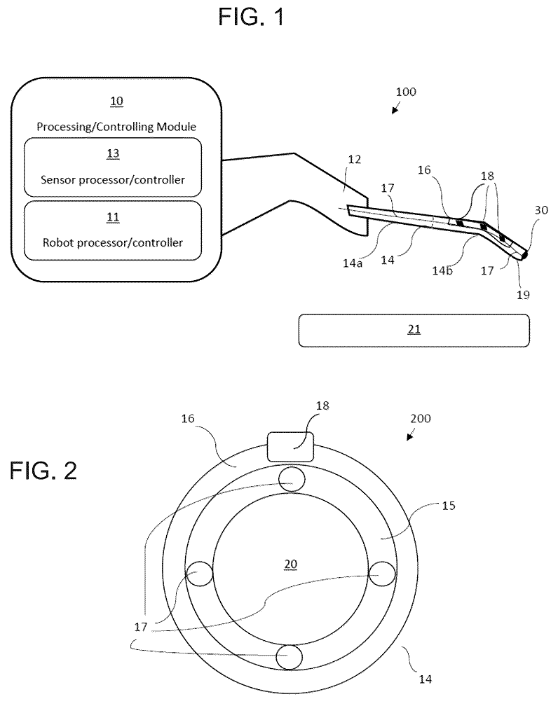

Reference is now made to FIG. 1, which is a schematic illustration of an interventional robotic system 100, according to some embodiments of the present disclosure. System 100 includes a processing/controlling module 10, an arm 12 and a flexible elongated device 14. For example, device 14 may include a steerable catheter, an endoscopic shaft, or another suitable interventional elongated flexible device. Elongated flexible device 14 may include a curve magnetometer sensor module 16, for example as described in U.S. Pat. No. 11,712,309. In some embodiments, curve sensor module 16 includes a plurality of magnetometer sensors 18, located in various locations along device 14. Additionally, system 100 may include a transmitter 21. Transmitter 21 may generate and/or transmit electromagnetic fields, which may be sensed by curve sensor module 16 and/or sensors 18.

Module 10 and/or arm 12 may be included in a surgical robot, and/or may be configured to perform surgical or interventional procedures. Processing/controlling module 10 may control how components of the surgical robot operate. For example, module 10 may control how arm 12 and/or elongated device 14 move and/or operate, for example how device 14 moves inside a lumen, to which it is inserted. Module 10 may include robot controller unit 11 and full curve tracking unit 13. Robot unit 11 and/or tracking unit 13 may include hardware and/or software components that may be encased wholly or partially together. In some embodiments, robot unit 11 and/or tracking unit 13 may be installed on separate devices, at least partially.

According to various embodiments of the present invention, information may be generated by tracking unit 13, for example based on data received from sensor module 16, and may be fed to robot unit 11, which may use the input from unit 13 to adjust how the surgical robot is controlled, for example to move and/or operate arm 12 and/or flexible elongated device 14. Module 10 may control steering of elongated device 14 by steering wires 17, which may pass along device 14 through a rim of device 14, from arm 12 to a distal tip 19 of device 14. System 100 may include at least two steering wires, and in some embodiments three or four steering wires 17.

Curve sensor module 16 may provide, to tracking unit 13, data about positions and/or orientations along device 14, and/or data that enables tracking unit 13 to calculate a fully localized curve shape of a respective section along device 14, for example based on the positions and/or orientations data. For example, curve sensor module 16 may provide data about locations and orientations along a distal portion of device 14. In some embodiments, curve sensor module 16 provides magnetic field readings from sensors 18, which may enable tracking unit 13 to calculate positions and/or orientations along device 14, for example relative to transmitter 21. Curve data may be represented for example as a plurality of 6-DOF or 5-DOF values along the device's curve, described for example using 3D position vectors and 2 or 3 Euler angles for the orientation, or quaternions for the orientation, or 4×4 3D transformations encoding both position and orientation, or using any other suitable method.

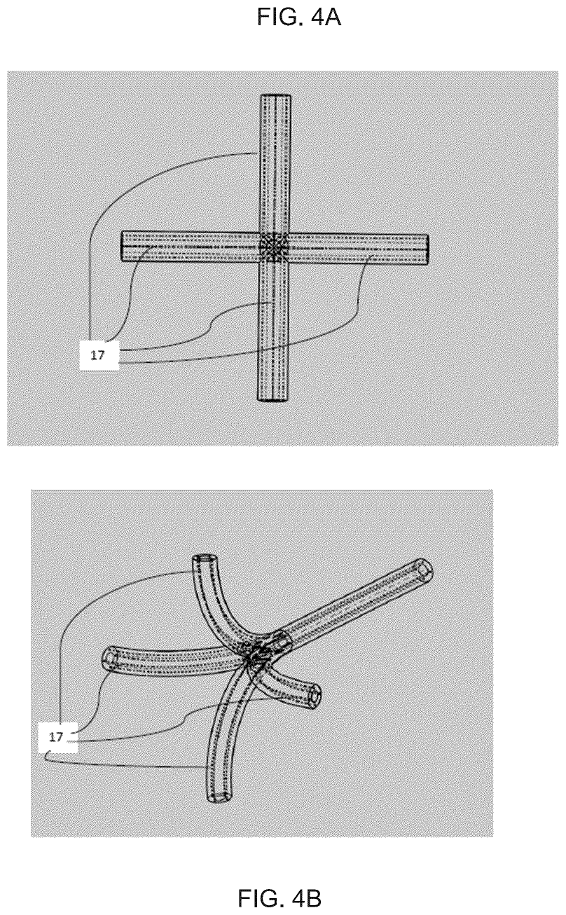

The fully localized curve shape, calculated by curve tracking unit 13, may encapsulate 6-DOF position and location data along at least a section of device 14. This data can be fed to robot control 11 as feedback input, based on which robot unit 11 may adjust the steering of arm 12 and/or device 14, for example by pulling and/or releasing each of wires 17 jointly or individually (see for example FIGS. 4A-4B).

In some embodiments of the present invention device 14 may include a camera 30 at tip 19. Image data may be provided from camera 30 to control 11 and/or may be used for further calibrating the control of steering wires 17.

For example, in a case of four steering wires 17, by pulling each wire device 14 bends in a certain direction out of four possible bending directions (see for example FIGS. 4A-4B). For example, four steering wires 17 include two pairs, wherein each two wires in each pair bend the endoscope in opposite directions. In this case, in order to bend the endoscope in a certain direction, a combination of wires can be pulled in certain amounts relative to an initial state of the wires. For example, as described in more detail herein, wires 17 may initially be in a zero-tension state. In some embodiments, while pulling a steering wire, the opposite wire is released as to allow device 14 to bend in the respective and/or desired direction without creating excessive tension on opposite wire while doing so. In other embodiments, the steering wires can be pulled to bend the device in a certain direction without releasing the opposite wires. This can be beneficial for example in cases where the device may slightly shrink in length while being bent. In some embodiments, a robot controller (or driver) maintains a constant tension on the pull-wires to make sure that no slack is formed on the wires, and that they're constantly tense.

Throughout the present disclosure, the term “pose” means a position along device 14, in which device 14 is posed, including a location and a curve along device 14.

While this example is given for a four pull-wire design with wires 17 evenly spaced around the circumference of the endoscope, it is to be understood that similar capabilities can be achieved with a three pull-wire design, four pull-wire design with non-evenly spaced wires, or any other suitable number and positioning of pull wires. In all cases a super-position of different tensioning of the wires enables deflection in all directions on the plain perpendicular to the axis of elongated device 14.

In some embodiments, control 11 may perform an initial calibration stage of wires 17 (called herein zero calibration). For example, in some embodiments the steering wires may have slack, such that pulling of a wire 17 may not immediately result in a deflection of device 14, as may be sensed by curve tracking unit 13. In some embodiments, control 11 brings steering wires 17 to a “zero tension” state, i.e. a state of initial tension where the wires are not loose, but still don't apply effective force on device 14. Controller 11 may bring wires 17 to a zero tension state by pulling wires 17 with a certain predetermined relatively low pulling force, which does not cause the device to substantially shrink or deflect at any direction, but essentially just eliminates any slack and/or looseness which may exist in the wires between robot controller 11 and device 14. Controller 11 may include, for example, pull-wire motors to control each wire 17, for example separately from the other wires 17.

When device 14 at a zero tension state, wires 17 are in a desired low tension, according to some embodiments. In some embodiments, when device 14 is at zero tension state, all single or combined steering actions, e.g. steering directions and amounts, are references to that zero-tension level, which leads to a repeatable deflection behavior of the device regardless of slack which may have existed prior to the zero-tension calibration in the steering wires. In another embodiment, the device's zero tension may be achieved in a wire displacement approach, rather than pull-force approach; each steering wire may be pulled slowly until the device starts to deflect in any direction, as sensed by curve tracking unit 13. At this state, the wire is assumed to be in zero tension state (because any further pull action of that wire causes the device to deflect). By repeating this process for each of the pull-wires, all pull-wires are brought to their zero-tension state and all further steering actions can be referenced to that zero-tension state.

In some embodiments of the present disclosure, the zero-tension process can be performed at the factory and/or during the manufacturing process of device 14. Device 14 can be supplied “pre-tensioned” with all slack eliminated. In some embodiments of this disclosure, the zero-tension process is performed immediately before or during an endoscopic and/or interventional procedure with device 14. One advantage of performing the zero-tension process during the procedure is that it allows the device to be manufactured and stored with slack on the wires, which simplifies the cost of production, eliminate the need for holding the wires in a known position, and eliminates the risk of internal forces which may occur between different parts of the device during shipping or storage due to the tension which may exist on the wires, or may be created through changing temperatures or forces that the device may experience during transportation and shipping between the zero-tension process and actual use.

The zero-tension calibration process can be performed while device 14 is straight along its length, or at a reset position. For example, zero-tension can be performed at the beginning of a robotic endoscopic procedure, when device 14 is mount to a robotic system and held straight, before entering a patient's body. Additionally or alternatively, the zero-tension calibration can be performed after entering the patient's body, for example, the patient's lung in a robotic bronchoscopy procedure. In this case, the zero-tension calibration can be performed while the device is in a straight pose inside the patient's trachea and before taking any further turns inside the patient's lung. The zero-tension process can be performed once or repeatedly during the procedure. Zero-tension of the pull-wires can be achieved with zero-tension calibration method as mentioned above, but can also be achieved using additional methods, as is further explained below.

In some embodiments, by remaining in zero-tension state throughout a robotic endoscopic procedure, regardless of the device's momentary deflected state (which may be caused by inserting the device into a curved lumen and/or which may affect the zero state of the wires, as explained in more detail below), a predictive deflection model, for example, can be trained and/or programmed to relate between pull-wire actions and device's deflection based on tracked curves which is more invariant of the device's current deflected state. For example, when the device is inserted into a curved lumen, such as a curved airway, it may be desired that the device remains loose such that it does not apply excessive force on surrounding tissue. To remain loose while inserted into a curved lumen, the device needs to be in a zero-tension state so that all pull-wires are loose, but without slack.

Staying in zero-tension state is potentially beneficial so that the device's distal deflectable section always stays straight if not intentionally bent, instead of bending in undesired bending directions due to overall bending of the device. According to some embodiments, this helps maintaining the softness of the device, to reduce forces which are applied by the device's deflectable section on the tissue. On the contrary, when zero-tension calibration is only done once (for example, in a reset state of the device, such as when entering the patient's lung), each further turn of the device may then shift the pull-wires zero-tension state such that the device is no longer in the same static zero-tension state. For example, in each turn inside the anatomy the device forms a bending at the proximal section of the device. Thus, for example, pull wires on the inner side of the bend experience shorter path than pull wires on the outer side of the bend. For example, this path length difference shifts the pull-wires zero-tension state.

When device 14 shifts the pull-wires zero-tension state it may, for example, get undesirably bent, apply excessive forces on surrounding tissues, become undesirably stiff, and/or other undesirable results. This may make a deflection calibration model less accurate, because it is referenced to a different zero-tension state of the device's pull-wires. A method for dynamically tracking the pull-wires zero-tension state based on tracked curves is explained in further detail below.

In some embodiments of the present invention, device 14 may include a deflectable section 14b and a non-deflectable section 14a. Robot unit 11 may execute steering actions that may deflect deflectable section 14b relative to non-deflectable section 14a, thus creating a bending angle between distal tip 19 of device 14 and non-deflectable section 14a.

Reference is now made to FIG. 2, which is a cross-sectional view 200 of device 14, according to some embodiments of the present disclosure. As described herein, in some embodiments, device 14 may include curve sensor module 16, for example having sensors 18, and steering wires 17 passing through its wall 15. In some embodiments, device 14 may include a working channel 20, through which various tools may be inserted, for example during an interventional procedure. Although four steering wires 17 are shown, various embodiments of the present invention may include different quantities of steering wires, for example two wires or three wires.

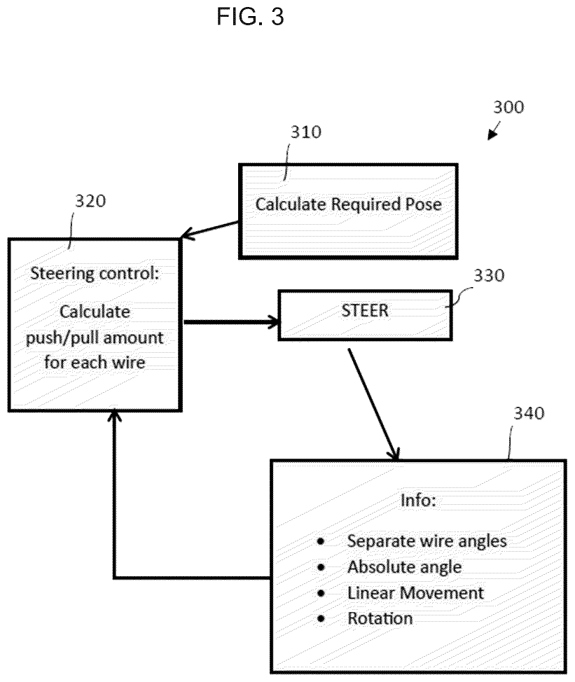

Reference is now made to FIG. 3, which is a flowchart illustrating a method 300 for controlling an interventional robotic system, according to some embodiments of the present disclosure.

As indicated in block 310, processing/controlling module 10 may calculate a required pose, for example based on received commands and/or based on received and/or generated three-dimensional image data and/or three-dimensional curve and/or position data. Processing/controlling module 10 may send the calculated required pose as a request to robot controller unit 11.

As indicated in block 320, robot controller unit 11 may calculate the steering actions required to bring device 14 to the requested pose. For example, unit 11 may calculate the amount in which each of steering wires 17 is to be pulled and/or pushed/released in order to achieve the required pose.

As indicated in block 330, robot controller unit 11 may steer device 14 according to the calculated actions and/or by executing the calculated actions.

According to various embodiments of the present disclosure, steering wires 17 are fixed at the distal tip 19. Robot controller unit 11 and/or controlling module 10 may pull a steering wire 17, for example to deflect deflectable section 14b in the direction of the pulled wire 17. For example, by pulling two adjacent wires 17 in a certain ratio, controller unit 11 may generate a deflection in a direction between the deflection directions of the two wires. Pulling a combination of two or three or more wires 17 may enable deflection in any direction, between 0 to 360 degrees. It may also affect the stiffness of the device. For example, in some embodiments, by pulling all four steering wires 17 the stiffness of the device increases.

According to some embodiments, the deflection of section 14b by wires 17 may require a pre-operational and/or real-time calibration and or tuning. For example, due to imperfections in the structure, components and/or connections inside of device 14, the output of pulling a wire 17 or a combination of wires 17 in order to achieve a deflection in a specific, predetermined direction may yield a deflection in a different direction. For example, pulling a wire 17 may cause a deflection in a distorted direction relative to the theoretical wire-corresponding direction wire.

Correction of the deflection direction may include calibrating the instructions of controller unit 11 to the actual deflection direction resulting from these instructions. For example, this calibration is performed for a plurality of directions, for example, all directions in intervals of 1 degree, 3 degrees or 5 degrees. In some cases, all wires can be pulled in different directions and amounts. For example, in some embodiments, each of steering wires 17 (for example, each of the two, three or four wires) is pulled by a certain amount, as shown for example in FIGS. 4A-4B. In some embodiments, each of the wires is pulled in displacement increments of known length, for example in increments of 0.1 mm or any other suitable amount, and the deflection direction is recorded for each wire configuration, as sensed by curve sensor module 16. In some embodiments, a wire 17 may be pulled separately in increments of a known length, and the deflection is calculated from the curve sensor module 16, for example, for each configuration of the pull wires. For example, once completed, controller 11 may assign and/or relate the output direction to the wire combination that generated it.

As indicated in block 340, tracking unit 13 may receive curve tracking data from curve tracking sensor module 16, such as data about positions and/or shape of device 14, along device 14. Based on the curve tracking data, tracking unit 13 may calculate and/or provide to robot controller unit 11 feedback input data, and/or robot controller unit 11 may calculate the feedback input data based on tracking data received from tracking unit 13. The feedback input data may be used by controller unit 11 as feedback to the steering actions, to adjust the steering for better accuracy, for example better similarity to the target required pose of device 14. For example, the feedback input data may include a three-dimensional bending angle (for example, two bending angles representing bend in each of two perpendicular planes) of distal tip 19 relative to non-deflectable section 14a and/or linear movement of device 14 in a lumen, for example relative to a previous time frame or a previous steering wires configuration. Controller unit 11 may identify, for example, that further pushing and/or pulling of individual wires 17 is required, in order to achieve a certain pose of device 14, and correct the steering accordingly. This feedback loop can be incorporated in a PID controller (Positional-Integral-Derivative controller), where the controlled parameters are the pull amounts of each steering wire, and the error feedback is the resulting deflection as sensed by the curve tracking, compared to the desired deflection.

In some embodiments, the robot may attempt to pull certain steering wires to reach the target required pose of device 14 based on a calibrated steering model. The model may be linear, cubic, polynomial or of any other suitable type. The model may convert between the desired 3D bending (angle and direction) of the deflectable section and weights and/or controlled parameters that determine the amount in which each of the steering wires needs to be pulled to achieve this deflection. When the robot pulls certain steering wires with the computed weights (based on the calibrated steering model), a certain mechanical deflection is actually performed, which may be different than the desired one. This may happen due to inaccurate steering model calibration, or imperfections in the steering model, or due to the fact that the device may be highly curved, which may change the force distribution as being applied by the steering wires, until reaching the distal deflectable section of the device, or due to any other reason. In any such case, the mechanical deflection which actually occurs in reality is sensed by the curve tracked device, with full position and shape data.

Since the curve sensor module 16 provides 6-DOF position and/or shape data and is fixed to the device, it is also fixed relative to the device's steering wires (at the device's tip), so that the measured deflection can be transformed to the steering wires coordinate system. With a 5-DOF sensor, which lacks roll data (such as an electromagnetic single-coil position sensor), the transformation between the 5-DOF sensor and the device's pull-wires cannot be achieved, because the sensor's roll is unknown and so it cannot be transformed to the device's pull-wires coordinate system, where the roll angle is of high importance. For example, with a 5-DOF sensor centered at the same origin of the device's pull-wires coordinate system, and with an unknown roll, a steering wire assigned to a certain direction axis can be mistakenly considered to be a steering wire of another direction axis, for example because the roll angle of a sensor 18 is unknown, and the roll angle of pull-wires 17 relative to the sensor 18 is unknown. This can result in erroneous steering actions when trying to achieve a certain specific deflection of the device. With a single 6-DOF sensor which includes roll data (such as an electromagnetic 3-coil position sensor), the transformation between the 6-DOF sensor and the device's pull-wires can be achieved. However, when a combination of pull-wires is pulled and the device is deflected, it is potentially hard or impossible to evaluate the device's actual deflection, because only a single point position and orientation (for example, tip position and orientation) is available, while the deflection is considered to be the distal shape of the device, i.e., the position and orientation of the tip relative to a more proximal point. Therefore, with a single sensor at the device's tip it is hard or impossible to evaluate the device's deflection and thus hard or impossible to close the loop between the steering actions and the resulting deflection. With a fiber-optic shape sensor, the shape of the fiber is known but the roll angle along the sensor might be missing, such that it is hard to transform between the fiber's sensed shape and the device's pull-wires. In some configurations, the fiber-optic shape sensor may provide roll data information, but then it may be integrated in a device such that it's not fixed relative to the tip and so it is hard or impossible to transform between the sensor's roll and shape to the pull-wires coordinate system. In some embodiments, one or more fiber-optic shape sensors are fixed to the device's tip to provide shape and roll data of the device's bendable section (deflectable section) in a coordinate system which is fixed relative to the device's tip, such that it can be transformed to the device's steering wires coordinate system. In this case, in some embodiments of the present disclosure, a fiber-optic shape sensor can be used to resolve between steering actions and device's deflections and can support the offline and/or online calibration between steering actions and device's deflections.

By using a 6-DOF curve sensor or a fiber-optic shape sensor which is fixed relative to the device's tip, in some embodiments, robot control 11 may perform a certain manipulation of the steering wires according to some vector \vec{ω} (which is a 4-dimensional vector in the case of four steering wires device or 2 or 3 dimensional vector in the case of two or three steering wires, respectively. In response to the manipulation, a certain tip deflection occurs at tip 19, which can be described as a micro deflection for example using a quaternion q in the steering wires coordinate system. The processor then collects samples of (\vec{ω}, q) pairs which can be used to train the steering model online/offline. The goal of the steering model is to be able to compute \vec{ω}(q), that is, to model the required steering wires action \vec{ω} based on the desired micro deflection q. As described above, with a full curve tracked device the desired tip deflection q can be computed in steering wires coordinates so that the model will be fed with coherent data. This is as opposed to a relative shape sensor such as an optical fiber shape sensor, where the sensed shape, and specifically, the position and orientation of the device's tip may be in some arbitrary, unknown roll, relative to the steering wires, or may slide relative to the device's tip and steering wires, as mentioned above.

By using a 6-DOF curve sensor or a fiber-optic shape sensor which is fixed relative to the device's tip, robot control 11 may perform a certain manipulation of the steering wires to deflect the device's tip in a certain desired direction to achieve a certain desired target pose of device 14. The desired target pose can be referenced to a camera 30, for example an endoscopic camera, which may be embedded in device 14, or to an external transmitter coordinate system, or to an anatomical structure inside which the device navigates, or to the device's pull wire coordinate system. Since the curve sensor module 16 is fixed relative to the device's tip, it is possible to convert between directions in the sensor's coordinate system and the pull wires coordinate system, as described herein. The same is possible with a fiber-optic shape sensor which is fixed relative to the device's tip, although this configuration may suffer from increased inaccuracy and/or jitter as explained in more detail herein.

According to some embodiments, the user may wish to deflect the device 14 in a direction relative to camera 30, for example, an endoscopic camera. For example, the user may display live video of an endoscopic camera, and the user may navigate inside an anatomical structure using the endoscopic camera. A user's left, right, up or down (or any other direction) steering commands may then need to result in a suitable device deflection action such that the resulting motion will show as left, right, up or down in the endoscopic camera. To achieve this, in some embodiments, the endoscopic camera may be embedded inside device 14, together with an embedded curve sensor module 16. A transformation between the tip sensor coordinate system and the camera can be calibrated before or during procedure, for example, using point-cloud registration methods, or using mechanical transformation matrices based on device's mechanical design or in any other suitable method. In some embodiments, when the user commands the robot to move (deflect) in a certain direction relative to the endoscopic camera, this direction is transformed to sensor tip coordinates (using the camera to sensor calibrated transformation) and robot control 11 can then predict the most suitable steering action (based on a prediction model as described herein) to deflect the robot in the desired direction.

In some embodiments, both the camera and the steering wires are fixed relative to each other, such that a steering model can be trained and/or programmed to transform between the pull wires and the camera coordinate system. However, without considering the device's current curved state, certain steering actions can result in unexpected deflection due to the device's current curved state. By using the device's current curved state, as tracked by curve sensor module 16, a better prediction model can be used to generate the best steering actions to achieve a desired motion relative to camera 30.

In some embodiments, the desired target pose of device 14 can be referenced to an anatomical structure. For example, in a navigational endoscopy, a robotic endoscope may be positioned in a certain pose inside the patient's anatomy. The user and/or system may then wish to deflect the scope to a different pose inside the anatomy, for example, so it will face an anatomical target (such as a lesion). To do so, a navigation system may localize the robotic endoscope inside a patient's anatomy using registration methods. In some embodiments, a robotic endoscope is localized inside a deforming anatomy using deformable and/or breathing registration methods. These may use curve sensor module 16 to provide registration between the curve-tracked device and the anatomy (which may be deformable and/or breathing). Additionally or alternatively, a fiber-optic shape sensor may be used to localized the device inside the anatomy, or a 5-DOF EM sensor, or a 6-DOF EM sensor or any other position and/or shape sensor. When the user and/or system wishes to deflect the device to a certain pose inside the anatomy, this pose needs to be represented in pull-wire coordinates, since otherwise the system will not know which wires need to be pulled in order to achieve the desired pose. To do so, the system needs to transform the desired device pose to pull-wire coordinates. This can be achieved with a curve sensor module 16 which is embedded inside device 14 or with a fiber-optic shape sensor which is fixed relative to the device's tip. By transforming the desired deflection pose to pull-wire coordinate system, robot control 11 can then predict the desired steering actions based on a calibrated steering model as described herein. Controlling module 10 may transform to pull-wire coordinates a device's tracked curve, calculated by module 16, the calculated tracked curve is localized in navigation coordinates, for example relative to transmitter 21. Without transforming the tracked curve to the pull-wire coordinates, erroneous steering actions may be performed which do not bring the device to the desired pose inside the anatomy, or do that with increased convergence time.

In some embodiments, being able to bring a robotic device 14 to a desired pose inside a patient's anatomy based on data from a curve sensor module 16 (or other suitable tracked sensors which are fixed relative to the pull-wires) is beneficial for autonomously, semi-autonomously or manually controlling a robotic device inside a patient's anatomy. In some embodiments, a navigation system may maintain a device's deflection to always face an anatomical target, which may be deforming and breathing. For example, the system may always wish to keep the device at a certain pose relative to the target, and may continuously correct the device's deflection by using the steering model based on feedback from a curve sensor. In some embodiments, the user may instruct a robotic navigation system to turn to a certain lumen (for example, airway), and the navigation system may perform the desired action in the anatomy based on the steering model and feedback from a curve sensor. By using a curve sensor which is fixed relative to the device's pull-wires and by utilizing a calibrated steering model, and combining this with a navigation system which localizes the tracked device inside a patient's anatomy, the device can then be effectively controlled and manipulated, manually or autonomously, relative to the patient's anatomy, which is highly valuable in navigational endoscopic procedures.

In some embodiments, \vec{ω}(q) may be modeled as a linear function, cubic function, polynomial, or using any other suitable model. In some cases, it may be easier to model q(\vec{ω}), that is, to predict the micro deflection which is about to occur on the device's tip based on some steering wires action \vec{ω}. In this case \vec{ω}(q) can be computed by inverse methods, for example, using non-linear optimization methods. While \vec{ω} and q are of course related, it may be the case that they depend on additional parameters. For example, as described above, the device's tip deflection may be affected by the device's state inside the body, for example, as it is curved inside a patient's lumen. As the device is curved, the force distribution applied by the steering wires may be changed such that different \vec{ω} needs to be applied in order to achieve deflection q. To overcome this, according to some embodiments, online training of the steering model as the device is being deflected (based on collected (\vec{ω}, q) pairs) may help in recalibrating the steering model to the device's current state. However, this can add time and frustration to the physician as they're trying to robotically steer the device to a certain pose (for example, while navigating inside a patient's body). For example, the device may not steer immediately to that pose, but rather converge to that pose in a trial-and-error convergence process.

According to some embodiments, a richer steering model may be trained which includes the device's current curved state as a parameter. For example, the deflection of the device's tip may be predicted based on the applied pulling of the steering wires \vec{ω}, as well as the device's current tracked curve (for example, in steering wires coordinates). q then becomes a function of all those parameters:

Predicted q=q(\vec{ω},rᵢ,qᵢ), where (rᵢ,qᵢ) may be 6-DOF positions and orientation describing the device's curve, as tracked by the curve sensor, for example, in steering wires coordinate system. q is then predicted not only based on the applied \vec{ω} (which may be a too simplistic model, as mentioned above), but also based on the device's current curved state which affects its tip deflection behavior. To train this richer model, according to some embodiments, training dataset may be collected based on steering wire actions \vec{ω}, on the device's tracked curve (rᵢ, qᵢ) and on the resulting micro deflection q, as measured by the curve sensor.

The richer model may be linear, cubic, polynomial or of any other suitable type. In some embodiments, a deep neural network may be used as a richer model. For example, the input to the neural network may be the device's current state and the steering wires action to apply (\vec{ω},rᵢ,qᵢ) and its output may be the predicted micro deflection q. The network may consist of several fully connected layers with non-linear activation (such as ReLU, Leaky ReLU, sigmoid or any other suitable non-linear activation functions). Its output may be a 4-dimensional unit vector representing the predicted micro deflection quaternion. When the system then desires to deflect the tip to a certain target pose q_{tgt} it will use the network to search for \vec{ω} such that q q(\vec{ω},rᵢ,qᵢ)=g_{tgt}. In some embodiments, instead of representing the device's micro deflection using a quaternion, the micro deflection can also be represented using two bending angles, such as two Euler angles, which represent the device's bend in two perpendicular bending planes. In this case q can be considered as a two-dimensional vector representing the device's deflection rather than a 4-dimensional quaternion.

In another embodiment, instead of feeding the neural network with the device's tracked curve as parameters (rᵢ,qᵢ) the network may “see” the device's curve by feeding it with a 3D input image, for example in a 3D-CNN (Convolutional Neural Network) architecture. For example, the system may generate a 3D patch containing a 3D image (in voxels) of the device's curve in space which may end for example at the patch's center (indicating the device's tip). The patch's coordinate system may be the tip's steering wires coordinate system (for example, such that the +/−X axis points to the “right”/“left” steering wire, the +/−Y axis points to the “top”/“bottom” steering wire respectfully, in a 4-wire configuration). The 3D-CNN neural network may also be fed, in a separate channel, with a 3D patch showing the desired device's curve after deflection. The CNN may then process the data to output a 4-dimensional vector representing the necessary steering action \vec{ω} to apply based on the device's current curved pose and the desired tip deflection. The computed steering action \vec{ω} is based on the device's current pose (as fed to the network in a 3D image), and the device's desired deflected pose (as also fed to the network in a 3D image). The neural network can “see” the current state, compare it with the desired deflected state, and generate a deeply intelligent decision \vec{ω} for the operation of the steering wires. The network may be trained by collecting samples of device states, steering actions and resulting states—generating the corresponding 3D images of each and using this collected data as training data for the network. Training may be performed offline, for example, by collecting data in a neutral setting, for example, by manipulating an endoscope in free space and recording the deflection vs. original state based on randomly applied \vec{ω} in random device curved poses, as being tracked by the curve sensor. Alternatively or additionally, training samples may be collected inside a patient's body using similar methods, which can provide for more realistic training samples (in a more realistic clinical terrain). Additionally, in some embodiments the neural network may be further trained and improved during procedure in an online training scheme, where training data is being collected during procedure and used to improve the offline-trained neural network.

Using a full curve sensor (shape and position) rather than a relative shape sensor (shape-only with no roll or without fixture to the device's tip) allows to generate more coherent data (in a relevant coordinate system) to a steering model. The steering model may be simple (e.g., linear, cubic, polynomial) or may be richer (e.g., a neural network) which can take the device's curved pose into account, as being tracked by the curve sensor.

In some embodiments, the feedback input includes the bending angle in each direction corresponding to each steering wire 17 relative to non-deflectable section 14a. In some embodiments, the feedback input includes a rolling angle of device 14. For example, in case system 100 includes two wires 17, achieving a certain pose may require pulling and/or pushing of wires 17 together with rolling of device 14 about its longitudinal axis.

Using the tracking data as feedback to the steering operations by controller unit 11, may enable controller 11 to learn, for example, by using a trained model, how to better calculate the required steering operations to achieve a certain pose of device 14, in a certain type of environment and/or of tissue.

For example, when device 14 is inside a certain type of lumen, controller unit 11 may decide to steer tip 19 to a certain direction. Accordingly, controller unit 11 may calculate steering actions that should make device 14 assume a corresponding pose. However, for example, interaction of device 14 with the surrounding tissue of the lumen may cause it to steer in a different manner, or may cause the required pose to change due to changes in the lumen shape, due to forces applied by device 14 on the surrounding tissue and vice versa. The tracking data, for example along with updated two- or three-dimensional image data about the surroundings of device 14, may enable controller unit 11 to refine the steering of device 14 in order to put tip 19 in the required direction.

In some embodiments, this process may include multiple iterations, until device 14 convergence into the required pose.

In some embodiments of the present invention, robot control unit 11 may rely on 6DOF positions and orientations of device 14, calculated by processor 10, for the steering, calibration, refinement and/or adjustment decisions. For example, the degree of freedom related to a roll angle of device 14 may be especially influential on prediction of how the steering wires should be adjusted.

In some cases, a fiber-optic shape sensor may be embedded in the device to sense the shape of a device, for example, an endoscopic device. As mentioned above, it may be hard or impossible to represent the sensed shape in the steering wires coordinate system and so the tracked shape might not be suitable for predicting the device's deflection based on pull-wire actions. In some embodiments, as mentioned above, a shape sensor may be fixed to the device's tip. Additionally, a shape sensor configuration can be chosen to also provide roll angles along the shape sensor, to result in a 6-DOF tracked curve which is fixed at the device's tip. In this case the tracked shape can be transformed to steering wires coordinate system which is necessary to predict deflection directions based on steering wires actions. However, by fixing the shape sensor to the device's tip alone, it is only the last most distal point of the shape that can be transformed to the pull-wires coordinate system, while more proximal points along the shape sensor may slide relative to the device's roll (twist) angle. This happens because the shape sensor may only be fixed at one or more points inside the device—for example, at the most distal (tip) and most proximal points. In this case, even if the shape sensor does provide full 6-DOF data rather than just 5-DOF shape which lacks the roll (twist) angle, the sensed shape cannot be fully transformed into the device's true roll (twist), and the twist distribution along the endoscopic device may not be accurately sensed by the shape sensor. The data provided by the shape sensor then lacks full twist information of the endoscopic device, which may impact the performance and/or accuracy of the deflection calibration and/or prediction model. In some embodiments, with a full curve sensor, such as an EM curve sensor, the sensor is embedded in the device and fixed all along the device. For example, in some embodiments the EM curve sensor is wrapped helically inside an endoscope's wall and fixed all along the endoscope, for example, using a heat shrink or glue points or reflow process. The sensor is then unable to slide or twist along the device, but is still able to bend with the device due to its helix nature. In this case, the device's full twist information is tracked by the 6-DOF curve sensor, and each point along the curve can be transformed to a corresponding pull-wire coordinate system with origin at that specific point. The tracked curve then provides valuable information about the device's current pose, which includes the device's full twist condition (at least along the deflectable portion of the device). This information can then be used to calibrate and/or predict the device's deflection based on steering actions using models which include the device's current pose (and full twist) as described above. By adding the full twist information along the device, the accuracy of the model is potentially improved, according to some embodiments of the present disclosure.

Throughout the disclosure calibration and prediction models were discussed, for correlation between pull-wire steering actions and desired device deflections or vice versa. For example, in some embodiments, these calibration models are used to perform an optimal steering action which leads to a desired deflection state of the device (with minimal trial and error iterations). In all such methods a steering action is performed, for example, which may comprise pulling and/or releasing a combination of pull-wires based on the desired deflection state and the device's current deflection state, as tracked by a curve sensor. Each steering action can be described by pulling or releasing a combination of pull-wires by certain amounts. The amounts may be displacements, for example, represented in millimeters, or they can be forces, for example, represented in Newton units. In each such case, it is potentially beneficial to start in a zero-tension state, so that all steering actions will be referenced to a zero-tension state, to increase their repeatability. For example, in the opposite case where some undefined slack exists in a certain pull wire, the wire can be pulled but no deflection may occur, due to the slack.

In some embodiments, static zero-tension can be achieved by performing a zero-tension calibration process at a certain reset state of the device, as mentioned above. However, as the device deflects (for example, proximally deflects with a bending radius of 30 mm, 40 mm or 50 mm or greater) its zero-tension state may shift due to asymmetrical distribution of the pull-wire lengths along the deflected device. In some embodiments, the zero-tension state of the device is being dynamically tracked using a curve sensor. By using the tracked curve of the device, the device's zero-tension shift can be predicted relative to the device's initial static zero-tension. The initial static zero tension calibration may be performed in a preprocedural and/or predetermined reset of the device, for example, when the device is in a straight position. To compensate for shifts in the device's zero-tension state, according to some embodiments of the present disclosure, the device's pull-wires can be pulled or released based on the device's current shape so that the pull-wires remain in zero-tension state even when the device is bent, as described in detail herein.

In some embodiments, zero-tension state can be dynamically maintained by keeping a low pulling force on all or some of steering wires, during the device's operation. In this case wires are never entirely released and are not allowed to build slack.

In some embodiments, repeated zero-tension calibration processes (as those described above) can be performed during a robotic endoscopic procedure.

In some embodiments, zero-tension state can be dynamically maintained by automatically performing zero-correction steering actions on all or some of steering wires, to keep them in a zero-tension state. Once the steering wires are put in a static zero-tension state using any zero-tension calibration method (for example, when the device is in its reset position at the beginning of a procedure) any further zero-tension state can be predicted based on the device's tracked curve (especially in the case of a full 6-DOF curve which is referenced to the device's pull-wires, as mentioned above). In some embodiments, a dynamic zero-tension calibration model can be learned which outputs zero-correction steering action for each tracked curve input. The model can be learned in methods similar to those mentioned above. For example, for each proximal or distal deflection of the device, a full zero-calibration process can be performed before procedure or during procedure and the resulting pull-wire states can be recorded. The process can be repeated in different poses, for example, manually or robotically, to collect samples of curved poses and corresponding pull-wire states, relative to a static zero-tension state which is performed prior to the data collection. With the collected pairs a model can be fitted or trained that converts between the device's curved pose to pull-wire states which reflect the shift in zero-calibration, according to some embodiments of the present disclosure.

In some embodiments, during procedure a static zero-tension calibration may be performed when the device is in zero state. In some embodiments, from that moment on the zero-tension dynamic calibration model can be used to keep track of the device's zero-tension state, by applying zero-correction steering actions based on the device's real-time tracked curve, as computed by the trained zero-tension dynamic calibration model.

In some embodiments, by tracking the device's dynamic zero-tension state the deflection model is improved (because it is referenced to a consistent zero-state), the device remains straight and soft during operation and applies less force on the surrounding tissue.

Throughout the disclosure, it is demonstrated how using curve tracking information improves the ability to deflect the device in specific desired directions with minimal trial and error iterations, as well as maintaining a device's dynamic zero-tension state during operation, which is beneficial for robotic endoscopy. It is shown that using other types of sensors, for example, which lack shape information, or which only contain relative shape data (for example, which lack exact roll or twist information, or which is unreferenced to the device's pull-wires) is potentially suboptimal for accurate robotic control.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A system for controlling an interventional robotic system, comprising:
a. an elongated flexible device, comprising:
i. a non-deflectable section in a proximal part of the device;
ii. a deflectable section in a distal part of the device;
iii. a curve sensor, configured to sense a curve of the deflectable section; and
iv. steering wires to deflect the deflectable section; and
b. a processing module comprising:
v. a curve tracking module; and
vi. a robot controller,
wherein the processing module is configured to:
calculate and perform steering actions, according to a current calibration state, by positioning each of the steering wires in a certain state, for bringing the device to a target pose;
receive curve tracking data from the curve tracking module; and
use the received curve tracking data as feedback to the robot controller, to determine a difference between a current pose of the device to the target pose of the device, and adjust the state of the steering wires to decrease the calculated difference.

2. The system of claim 1, wherein the steering wires are calibrated to achieve a zero-tension state, before a procedure or during a procedure.

3. The system of claim 2, wherein the steering wires are calibrated dynamically during a procedure, to achieve and maintain a static zero-tension state.

4. The system of claim 1, wherein the robot controller performs a closed-loop control of the device tip deflection.

5. The system of claim 1, wherein the robot controller uses a predictive model of deflection, to control the device tip deflection.

6. The system of claim 1, wherein the deflection control is dynamically calibrated.

7. The system of claim 1, wherein the device includes at least two steering wires.

8. The system of claim 1, wherein the device includes four steering wires.

9. The system of claim 1, wherein the curve tracking module computes the curve by using a 6DOF electromagnetic curve sensor.

10. The system of claim 1, wherein the curve tracking module computes the curve by using an optical fiber sensor fixed at a distal tip of the device.

11. A method for controlling an interventional robotic system, comprising:
a. calculating and performing by a robot controller steering actions, according to a current control calibration state, by positioning each of a plurality of steering wires in a certain state, for bringing a device to a target pose;
b. receiving curve tracking data of the device from a curve tracking module; and
using the received curve tracking data as feedback to the robot controller, to determine a difference between a current pose of the device to the target pose of the device, and adjust the state of the steering wires to decrease the calculated difference.

12. The method of claim 11, wherein the steering wires are calibrated to achieve a zero-tension state, before a procedure or during a procedure.

13. The method of claim 12, wherein the steering wires are calibrated dynamically during a procedure, to achieve and maintain a static zero-tension state.

14. The method of claim 11, wherein the robot controller performs a closed-loop control of the device tip deflection.

15. The method of claim 11, wherein the robot controller uses a predictive model of deflection, to control the device tip deflection.

16. The method of claim 11, wherein the deflection control is dynamically calibrated.

17. The method of claim 11, wherein the device includes at least two steering wires.

18. The method of claim 11, wherein the device includes four steering wires.

19. The method of claim 11, wherein the curve tracking module computes the curve by using a 6DOF electromagnetic curve sensor.

20. The method of claim 11, wherein the curve tracking module computes the curve by using an optical fiber sensor fixed at a distal tip of the device.
